(19) United States
(12) Patent Application Publication
(10) Pub. No.: US 2004/0230209 A1
(43) Pub. Date: Nov. 18, 2004

(54) Vascular anastomosis apparatus

(76) Inventor: Saqib Masroor, Greenville, NC (US)

Correspondence Address: Mark Terry, 227 Michigan Av. #204, Miami Beach, FL, 33139, US

(21) Appl. No.: 10/746,104

(22) Filed: Dec. 26, 2003

Related U.S. Application Data
(60) Provisional application No. 60/471,026, filed on May 16, 2003.

(51) Int. Cl. A61B 17/08

(52) U.S. Cl. 606/153

(57) ABSTRACT

An apparatus for facilitating anastomosis of a first tubular element with a second tubular element is disclosed. The apparatus comprises a ring for placement inside of the first tubular element such that the ring and the first tubular element are concentric. The apparatus further comprises a circular strap for placement around the second tubular element such that the circular strap and the first tubular element are concentric, wherein a first end of the first tubular element is placed inside of a first end of the second tubular element such that the ring and the circular strap are concentric. The apparatus further comprises an adjustment element coupled to the circular strap for adjusting the circular strap to apply pressure onto the ring and secure the first end of the first tubular element to the first end of the second tubular element.

### CROSS-REFERENCED APPLICATIONS

This U.S. patent application claims priority to U.S. Provisional Patent Application No. 60/471,026, entitled “Vascular Anastomotic Coupler” and filed by Saqib Masroor on May 16, 2003. The aforementioned U.S. Provisional Patent Application is hereby incorporated by reference in its entirety.

### PARTIAL WAIVER OF COPYRIGHT

All of the material in this patent application is subject to copyright protection under the copyright laws of the United States and of other countries. As of the first effective filing date of the present application, this material is protected as unpublished material. However, permission to copy this material is hereby granted to the extent that the copyright owner has no objection to the facsimile reproduction by anyone of the patent documentation or patent disclosure, as it appears in the United States Patent and Trademark Office patent file or records, but otherwise reserves all copyright rights whatsoever.

### FIELD OF THE INVENTION

This invention generally relates to the field of anastomotic surgery and more specifically to a device for facilitating vascular anastomotic surgery.

### DESCRIPTION OF RELATED ART

Replacement of native arteries with prosthetic grafts is a commonly performed procedure today, for a variety of different indications in many different anatomical regions of the human body. This includes repair of aortic aneurysms, less common non-aortic aneurysms, aortoiliac occlusive disease, upper and lower extremity revascularizations, brachiocephalic and other arch vessel reconstruction and mesenteric revascularizations.

The incidence of abdominal aortic aneurysms has been reported to be 21 per 100,000 person-years (Bickerstaff L K, Hollier L H, Van Peenen H J, et al: Abdominal aortic aneurysms: The changing natural history. J Vasc Surg 1984;1:6) and that of thoracic aneurysms up to 10.4, per 100,000 person-years (Clouse W D, Halleft J W Jr., Schaff H V, et al. Improved prognosis of thoracic aortic aneurysms: a population based study. JAMA 280 (22): 1926-9,1998). Abdominal aortic aneurysms (AAA) are associated with the highest risk of rupture of up to 50% per year once they reach a size of 8 cm, a condition that carries a mortality of 50% in people who make it to the hospital. It should be noted that half of the people with ruptured abdominal aneurysms die before reaching the hospital. Ruptured AAA is the 15th leading cause of death in men in the United States (Cronenwett J L, Krupski W C and Rutherford R B. Abdominal aortic and iliac aneurysms).

To prevent this catastrophic event, prophylactic repair of abdominal aortic aneurysms is performed once they reach a critical diameter. In the United States alone, 40,000 AAA repairs are performed annually and this procedure is one of the most common vascular surgery procedures performed (Gillum R F. Epidemiology of aortic aneurysm in the United States. J Clin Epidemiol 1995;48:1289). The conventional treatment involves replacement of the an urysmal (dilated) aorta with a prosthetic graft. In textile or fabric grafts such as Dacron grafts, the basic polymer is made into a yarn, which is then knit or woven into a graft (Brewster D C. Prosthetic Grafts. In Rutherford (Ed) Vascular Surgery. 5th Edition. W. B. Saunders Company, Philadelphia, 2000). In case of non-textile grafts such as expanded polytetrafluoroehtylene (ePTFE) grafts, the technique of precipitation of the polymer from solution of the material is used for construction of the graft (see Brewster D C). The grafts are either straight (for aorti-iliac anastomosis) or bifurcated (for aorto-bifemoral anastomosis).

In light of the above, proper execution of the anastomotic procedure is crucial to the success of the surgery. When an anastomosis is constructed between two blood vessels or a graft and a blood vessel, the standard technique involves sewing the two ends together with a suture and needle. While the two ends are being sewn together, the two ends on either side are clamped and there is no blood flow across the area of the anastomosis. In addition to being time-consuming, it is also prone to leaking from the suture line.

Suture-line bleeding, while usually manageable, can have disastrous consequences, especially in the older population whose arteries have thick walls and are calcified. Bleeding from such arteries is not easily controlled with additional hemostatic stitches. These hemostatic stitches are placed at the bleeding points in the suture line once the clamps are removed and blood is allowed to flow across the anastomosis. In inexperienced hands, these stitches can be a cause of more trouble because of the less than ideal conditions under which they are placed. It is important to have a successful anastomosis on the first attempt and complete it in a timely fashion.

Therefore a need exists to overcome the problems with the prior art as discussed above, and particularly for a way to perform vascular anastomotic surgery more efficiently.

### SUMMARY OF THE INVENTION

The present invention, according to a preferred embodiment, overcomes problems with the prior art by providing an efficient and easy-to-implement apparatus for performing vascular anastomosis surgery.

A method and apparatus for performing an anastomotic surgery is disclosed. In an embodiment of the present invention, the apparatus for facilitating anastomosis of a first tubular element with a second tubular element includes a ring for placement inside of the first tubular element such that the ring and the first tubular element are concentric. The apparatus further includes a circular strap for placement around the second tubular element such that the circular strap and the first tubular element are concentric, wherein a first end of the first tubular element is placed inside of a first end of the second tubular element such that the ring and the circular strap are concentric. The apparatus further includes an adjustment element coupled to the circular strap for adjusting the circular strap to apply pressure onto the ring and secure the first end of the first tubular element to the first end of the second tubular element.

In another embodiment of the present invention, the apparatus further includes at least one spike coupled to the outside diameter of the ring, wherein when the ring is placed inside of the first tubular element, the at least one spike extends through a surface of the first tubular element so as to secure the ring inside of the first tubular element.

In an embodiment of the present invention, the apparatus for facilitating anastomosis of a first tubular element with a second tubular element includes a ring for placement inside of the first tubular element such that the ring and the first tubular element are concentric. The apparatus further includes at least one adjustable spike coupled to the outside diameter of the ring, wherein when the ring is placed inside of the first tubular element, the at least one spike extends through a surface of the first tubular element so as to secure the ring inside of the first tubular element and wherein the location of the at least one adjustable spike on the outside diameter of the ring can be adjusted. The apparatus further includes a circular strap for placement around the second tubular element such that the circular strap and the first tubular element are concentric, wherein a first end of the first tubular element is placed inside of a first end of the second tubular element such that the ring and the circular strap are concentric. The apparatus further includes an adjustment element coupled to the circular strap for adjusting the circular strap to apply pressure onto the ring and secure the first end of the first tubular element to the first end of the second tubular element.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings.

FIG. 1 is an illustration of a frontal view of a ring of one embodiment of the present invention.

FIG. 2 is an illustration of an angled view of the ring of FIG. 1.

FIG. 3 is an illustration of a frontal view of a circular strap of one embodiment of the present invention.

FIG. 4 is an illustration of an angled view of the circular strap of FIG. 3.

FIG. 5 is an illustration of an angled view of the ring being inserted into the circular strap.

FIG. 6 is an illustration of an angled view of the ring presently inserted into the circular strap.

FIG. 7 is an illustration of an angled view of the ring presently inserted into the lumen of a first vascular element in one embodiment of the present invention.

FIG. 8 is an illustration of an angled view of the first vascular element being inserted into the lumen of a second vascular element in one embodiment of the present invention.

FIG. 9 is an illustration of an angled view of the circular strap presently placed around the second vascular element in one embodiment of the present invention.

FIG. 10 is an illustration of a side view of a ring in a second embodiment of the present invention.

FIG. 11 is an illustration of an angled view of the ring of FIG. 10.

FIG. 12 is an illustration of an angled view of a set of loops for placing around the ring of FIG. 10 in one embodiment of the present invention.

FIG. 13 is an illustration of a side view of the set of loops of FIG. 12.

FIG. 14 is an illustration of an angled view of the set of loops of FIG. 12 in an interlaced arrangement.

FIG. 15 is an illustration of a side view of the set of loops of FIG. 12 in an interlaced arrangement.

FIG. 16 is an illustration of an angled view of the ring of FIG. 10 coupled with the set of loops of FIG. 12.

FIG. 17 is an illustration of a side view of the ring of FIG. 10 coupled with the set of loops of FIG. 12.

FIG. 18 is an illustration of a cross sectional view of the top side of FIG. 17.

FIG. 19 is an illustration of a cross sectional view of the bottom side of FIG. 17.

### DETAILED DESCRIPTION OF THE PREFERRED EMBODIMENTS

It should be understood that these embodiments are only examples of the many advantageous uses of the innovative teachings herein. In general, statements made in the specification of the present application do not necessarily limit any of the various claimed inventions. Moreover, some statements may apply to some inventive features but not to others. In general, unless otherwise indicated, singular elements may be in the plural and vice versa with no loss of generality. In the drawing like numerals refer to like parts through several views.

The present invention, according to a preferred embodiment, overcomes problems with the prior art by providing multi-project buffer management for an efficient and easy-to-implement multi-project management system utilizing the critical chain methodology.

### First Embodiment

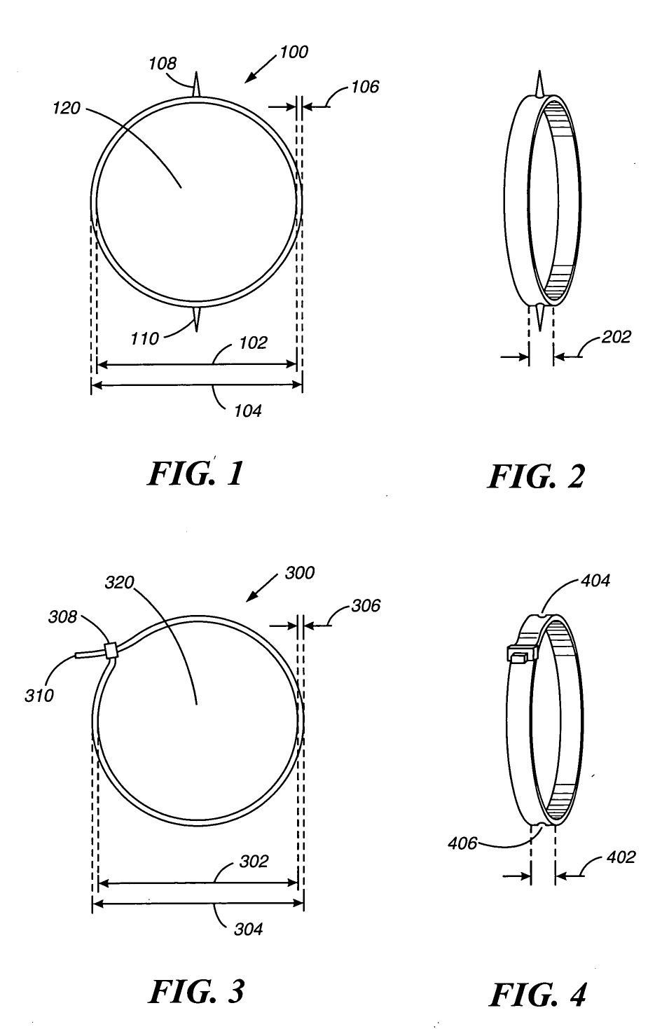

FIG. 1 is an illustration of a frontal view of a ring 100 of one embodiment of the present invention. FIG. 1 shows the ring 100 having an outside diameter 104 and an inside diameter 102. The ring 100 must be placed inside of the lumen of a tubular vascular element such as a vein, an artery, any other type of blood vessel or a prosthetic graft. This is described in greater detail below. Thus, the outside diameter 104 of the ring 100 must accommodate the inside diameter of the lumen of the vascular element in which the ring 100 is placed, such that the ring 100 fits securely within the lumen. That is, the outside diameter 104 of the ring 100 is substantially the same as, or slightly smaller than, the inside diameter of the lumen of the vascular element in which the ring 100 is placed. Thus, the outside diameter 104 of the ring 100 can be from about 6 mm to about 45 mm, depending on the vascular element in which the ring 100 is placed.

FIG. 1 further shows a lumen 120 of the ring 100, which must be substantially wide to allow a proper amount of blood to flow through the ring 100. For this reason, the inside diameter 102 of the ring 100 can be from about 6 mm to about 36 mm. FIG. 1 also shows the surface of the ring 100 having a thickness 106. The thickness 106 of the surface of the ring 100 can be from about 0.5 mm to about 3 mm.

Additionally, the ring 100 includes a spike 108 located on the top of the outside circumference of the ring 100 and a spike 100 located on the bottom of the outside circumference of the ring 100. Both spikes 108 and 110 are coupled to the outside surface of the ring 100 and extend radially from the center of the lumen 120 of the ring 100. The spikes 108 and 110 are sufficiently sharp to pierce the vascular element in which the ring 100 is placed. Further, the length of spike 108 and 110 can be from about 3 mm to about 8 mm. The thickness of spike 108 and spike 110 at their base can be from about 0.5 mm to about 5 mm.

FIG. 1 shows that spikes 108 and 110 are located 180 degrees apart from each other around the circumference of the ring 100. In another embodiment of the present invention, spikes 108 and 110 are located any number of degrees apart from each other around the circumference of the ring 100. It should be noted that although FIG. 1 shows two spikes, the present invention supports any number of spikes located any number of degrees apart from each other around the circumference of the ring 100.

The variable positioning of spikes in any location around the circumference of the ring 100 is advantageous because it allows the spike(s) to be specifically positioned in places where they are needed. There are occasions when the vascular element in which the ring 100 is placed is not healthy or structurally sound in certain areas. These diseased or damaged areas would not allow a spike to pierce through the surface without tearing or further damaging the surface area. By allowing the variable positioning of the spikes around the ring 100, diseased or damaged surface areas of the vascular element can be avoided and the spikes can be concentrated on healthy and/or structurally sound surface areas that would allow a spike to pierce the surface and secure the ring 100 within the lumen of the vascular element.

In an embodiment of the present invention, the ring 100 is composed of stainless steel, titanium, carbon fiber, an alloy or any other material suitable for inserting into an artery. The ring 100 may also be composed of a material that does not interact with blood, such as pyrolitic carbon.

FIG. 2 is an illustration of an angled view of the ring of FIG. 1. FIG. 2 shows that the ring 100 has a depth 202. The depth 202 of the ring 100 can be from about 3 mm to about 10 mm.

FIG. 3 is an illustration of a frontal view of a circular strap 300 of one embodiment of the present invention. FIG. 3 shows the circular strap 300 having an outside diameter 304 and an inside diameter 302. The circular strap 300 is placed around a tubular vascular element such as a vein, an artery, any other type of blood vessel or a prosthetic graft. This is described in greater detail below. Thus, the inside diameter 302 of the circular strap 300 must accommodate the outside diameter of the vascular element around which the circular strap 300 is placed, such that the circular strap 300 fits securely around the vascular element. That is, the inside diameter 302 of the circular strap 300 is substantially the same as, or slightly larger than, the outside diameter of the vascular element around which the circular strap 300 is placed. Thus, the inside diameter 302 of the circular strap 300 can be from about 6 mm to about 45 mm, depending on the vascular element around which the circular strap 300 is placed.

Note that in addition to being placed around a vascular element, the circular strap 300 is also placed around the ring 100 (see FIG. 6 below). For this reason, the inside diameter 302 of the circular strap 300 must accommodate the outside diameter 104 of the ring 100 around which the circular strap 300 is placed, such that the circular strap 300 fits securely around the ring 100. That is, the inside diameter 302 of the circular strap 300 is substantially the same as, or slightly larger than, the outside diameter 104 of the ring 100, around which the circular strap 300 is placed.

FIG. 3 further shows a lumen 320 of the circular strap 300, which must be substantially wide to allow a vascular element to fit within it. For this reason, the inside diameter 302 of the circular strap 300 can be from about 6 mm to about 45 mm. FIG. 3 also shows the surface of the circular strap 300 having a thickness 306. The thickness 306 of the surface of the circular strap 300 can be from about 0.5 mm to about 3 mm.

Additionally, the circular strap 300 includes an adjusting element 308 that allows the circular strap 300 to be adjusted in size. The circular strap 300 consists of an elongated strap or belt element having a first end at the adjusting element 308 and a second end 310. The second end 310 is inserted into the adjusting element 308. As the second end 310 is pulled father and farther through the adjusting element 308, the lumen 320 of the circular strap 300 becomes smaller. In this way, the circular strap 300 is tightened around a vascular element in order to secure the circular strap 300 onto the vascular element.

In one embodiment of the present invention, the adjusting element 308 only allows the end 310 to travel through the adjusting element 308 unidirectionally. That is, the adjusting element 308 only allows the end 310 to travel through the adjusting element 308 to the left of FIG. 3 or in the tightening direction. The adjusting element 308 does not allow the end 310 to travel through the adjusting element 308 to the right of FIG. 3 or in the loosening direction. In on embodiment of the present invention, the adjusting element 308 is a buckling element, a ratcheting element or a screwing element. Any of these elements perform the same functions as adjusting element 308, which only allows the end 310 to travel through the adjusting element 308 unidirectionally so as to secure the circular strap 300 onto the vascular element.

FIG. 4 is an illustration of an angled view of the circular strap 300 of FIG. 3. The circular strap 300 includes a circular orifice 404 located through the top of the surface of the circular strap 300 and a circular orifice 406 located through the bottom of the surface of the circular strap 300. Both orifices 404, 406 extend through the outside surface of the circular strap 300 and extend radially from the center of the lumen 320 of the circular strap 300. Note that the orifices 404, 406 are located in similar locations as the spikes 108, 110, such that when the circular strap 300 is placed around the ring 100, the orifices 404, 406 and the spikes 108, 110 are aligned. The orifices 404, 406 are sufficiently wide to allow the spikes 108, 110 to extend through the orifices 404, 406, respectively. The width of orifices 404, 406 can be from about 0.5 mm to about 6 mm.

FIG. 4 shows that orifices 404, 406 are located 180 degrees apart from each other around the circumference of the circular strap 300. In another embodiment of the present invention, orifices 404, 406 are located any number of degrees apart from each other around the circumference of the circular strap 300. It should be noted that although FIG. 4 shows two orifices, the present invention supports any number of orifices located any number of degrees apart from each other around the circumference of the circular strap 300, so as to correspond and align with spikes located around the circumference of the ring 100 when the circular strap 300 is placed around the ring 100.

FIG. 4 further shows that the circular strap 300 has a depth 402. The depth 402 of the circular strap 300 is substantially similar to the depth 202 of the ring 100. Thus, the depth 402 of the circular strap 300 can be from about 2 mm to about 10 mm.

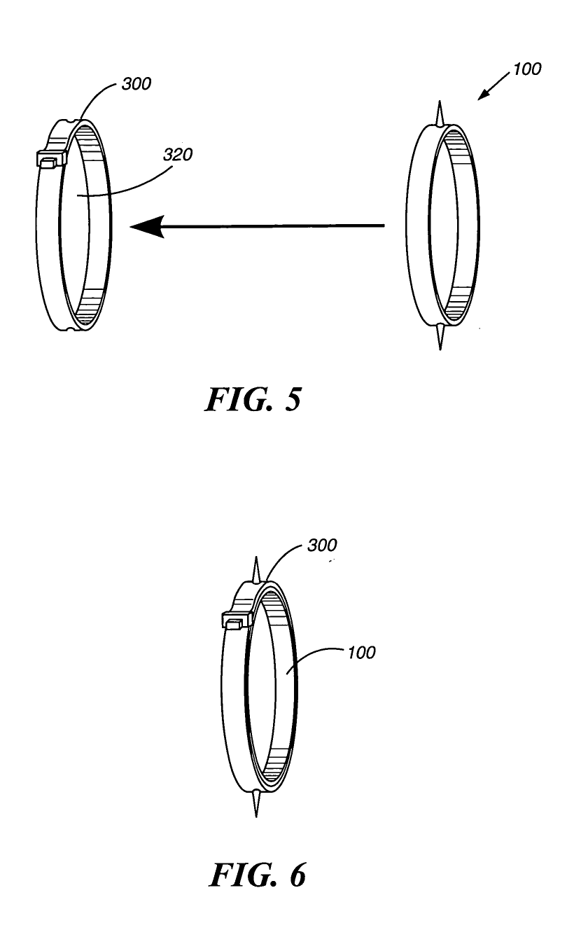

FIG. 5 is an illustration of an angled view of the ring 100 being inserted into the circular strap 300. Note the ring 100 is inserted into the lumen 320 of the circular strap 300. FIG. 6 is an illustration of an angled view of the ring 100 presently inserted into the circular strap 300. Note the inside diameter 302 of the circular strap 300 accommodates the outside diameter 104 of the ring 100 around which the circular strap 300 is placed, such that the circular strap. 300 fits securely around the ring 100. The inside diameter 302 of the circular strap 300 is thus substantially identical to, or slightly larger than, the outside diameter 104 of the ring 100. When the circular strap 300 is placed around the ring 100, the spikes 108, 110 extend through the orifices 404, 406, respectively. This holds the ring 100 securely within the lumen 320 of the circular strap 300.

An advantage of the present invention is that once the circular strap 300 has been placed around the ring 100, it is tightened and thus compresses the walls of the two vascular elements down on to the ring 100. The tightening motion of the circular strap 300 exerts a circumferential pressure on the entire anastomosis and seals the anastomosis when adequate pressure is applied. Note that a seal is obtained around the entire circumference of the anastomosis and not just on a finite number of locations. The amount of pressure that must be applied is variable and can be translated into a defined metric that is used when adjusting the circular strap 300 using the adjusting element 308.

### Execution of the Anastomosis Surgery

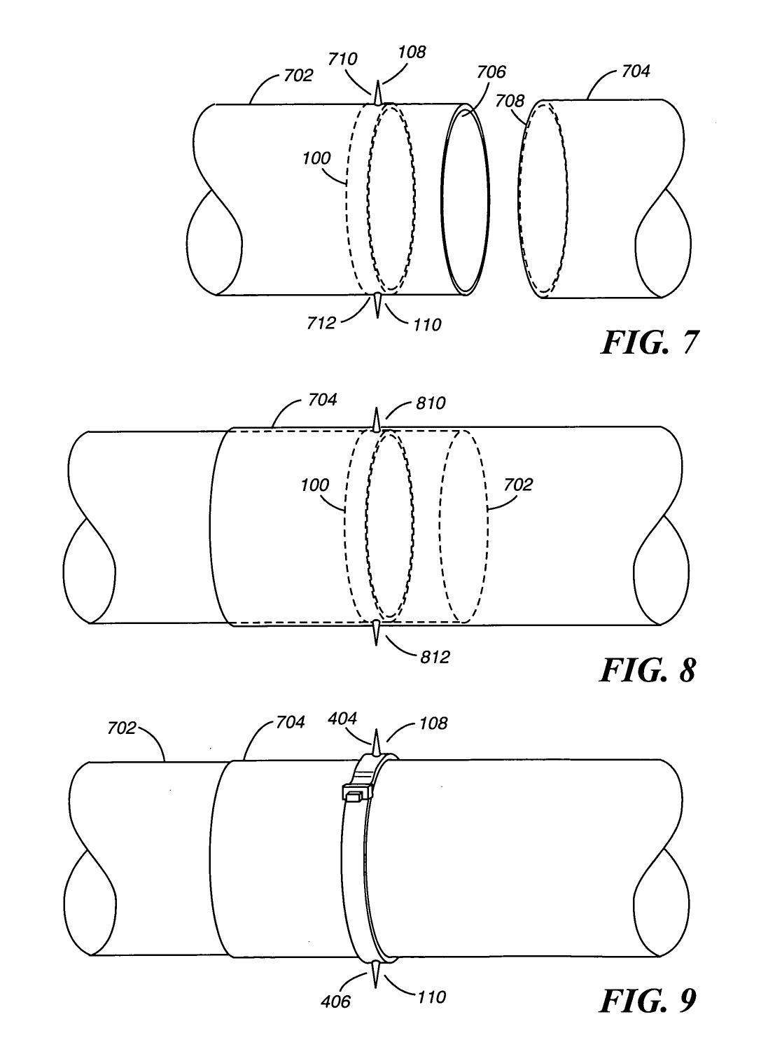

Generally, a vascular anastomotic surgery using the present invention begins with cleaning the adventitia of the proximal and distal anastomotic surfaces (herein referred to as vascular elements). Then, the inner diameter of a first vascular element is determined, in order to determine the diameter of the ring 100 to be used. Next, the ring 100 is placed inside the first vascular element. This is shown in FIG. 7.

FIG. 7 is an illustration of an angled view of the ring 100 presently inserted into the lumen 706 of a first tubular vascular element 702 in one embodiment of the present invention. FIG. 7 shows two tubular vascular elements 702 and 704 upon which an anastomosis surgery, otherwise known as an anastomotic coupling, shall be performed. As explained above, a tubular vascular element is as a vein, an artery, any other type of blood vessel or a prosthetic graft.

The ring 100 has been inserted into the lumen 706 of the vascular element 702 via a first or open end. The ring 100 is placed concentrically within the tubular vascular element 702. Note that the ring 100 is positioned about 3 mm from the open end of the vascular element 702. The spike 108 pierces and protrudes through the surface of the vascular element 702 at location 710 and spike 110 pierces and protrudes through the surface of the vascular element 702 at location 712, so as to hold the ring 100 securely within the lumen 706 of the vascular element 702. The ring 100 can be positioned within the lumen 706 of the vascular element 702 such that the spikes 108, 110 pierce through healthy and/or structurally sound surface areas that would allow a spike to pierce the surface and secure the ring 100 within the lumen 706 of the vascular element 702. The ring 100 can be adjusted so as to avoid diseased or damaged surface are as of the vascular element 702.

Note the outside diameter 104 of the ring 100 accommodates the inside diameter of the lumen 706 of the vascular element 702 in which the ring 100 is placed, such that the ring 100 fits securely within the lumen 706. Thus, the outside diameter 104 of the ring 100 is substantially identical to, or slightly smaller than, the inside diameter of the lumen 706 of the vascular element 702.

In one embodiment of the present invention, the diameter of the lumen 706 is enlarged such as by changing the shape of the vascular element 702 to an oval by squeezing it, so as to allow the ring 100 to be inserted into the lumen 706 of the first vascular element 702. That is, the vascular element 702 is squeezed gently by a surgeon's fingers in order to increase the diameter of the lumen 706 along one axis and allow the ring 100 to fit within the lumen 706. This allows the surgeon to insert the ring 100 into the lumen 706 such that the axis of the spikes 108, 110 lie along the enlarged diameter of the lumen 706. Once the ring 100 has been inserted into the vascular element 702 such that the ring 100 is concentric with the tubular vascular element 702, the vascular element 702 is returned to its normal circular shape, which allows the spikes 108, 110 to pierce through the surface area of the vascular element 702 and hold the ring 100 securely within the lumen 706.

The lumen 708 of the vascular element 704 awaits the insertion of the vascular element 702. FIG. 8 is an illustration of an angled view of the first vascular element 702 being inserted into the lumen 708 of a second vascular element 704 in one embodiment of the present invention.

Note the outside diameter of the first vascular element 702 accommodates the inside diameter of the lumen 708 of the second vascular element 704 in which the first vascular element 702 is placed, such that the first vascular element 702 fits securely within the second vascular element 704. Thus, the outside diameter of the first vascular element 702 is substantially identical to, or slightly smaller than, the inside diameter of the lumen 708 of the second vascular element 704. Alternatively, the outside diameter of the first vascular element 702 is identical to, or slightly larger than, the inside diameter of the lumen 708 of the second vascular element 704. Since a vascular element such as a vein or artery exhibits elastic qualities (such as being able to stretch or being collapsible and/or pliable), if the outside diameter of the first vascular element 702 is identical to, or slightly larger than, the inside diameter of the lumen 708 of the second vascular element 704, the first vascular element 702 could be folded, compressed or otherwise made smaller before insertion into the second vascular element 704. Further, the second vascular element 704 could be stretched, elongated or otherwise made larger before insertion of the first vascular element 702.

Note that the ring 100 is positioned about 3 mm from the open end of the vascular element 704. FIG. 8 also shows that spike 108 pierces and protrudes through the surface of the vascular element 704 at location 810 and spike 110 pierces and protrudes through the surface of the vascular element 704 at location 812, so as to hold the second vascular element 704 securely around the first vascular element 702. The ring 100 can be positioned within the lumen 708 of the vascular element 704 such that the spikes 108, 110 pierce through healthy and/or structurally sound surface areas that would allow a spike to pierce the surface and secure the ring 100 within the lumen 708 of the vascular element 704. The ring 100 can be adjusted so as to avoid diseased or damaged surface areas of the vascular element 704.

FIG. 9 is an illustration of an angled view of the circular strap 300 presently placed around the second vascular element 704 in one embodiment of the present invention.

Note the inside diameter 302 of the circular strap 300 accommodates the outside diameter of the vascular element 704 around which the circular strap 300 is placed, such that the circular strap 300 fits securely around the vascular element 704. The inside diameter 302 of the circular strap 300 is substantially identical to, or slightly larger than, the outside diameter of the vascular element 704. Further, when the circular strap 300 is placed around the vascular element 704, the spikes 108, 110 extend through the orifices 404, 406, respectively. This holds the vascular element 704 securely within the lumen 320 of the circular strap 300.

Subsequently, the adjusting element 308 on the circular strap 300 is used to tighten the circular strap 300 around the vascular element 704 in order to secure the circular strap 300 onto the vascular element 704. As explained above, the adjusting element 308 only allows the end 310 to travel through the adjusting element 308 unidirectionally.

Also note that the inside diameter 302 of the circular strap 300 accommodates the outside diameter 104 of the ring 100 around which the circular strap 300 is placed, such that the circular strap 300 fits securely around the ring 100.

An advantage of th present invention is the simplicity of the mechanism. Once learned by those implementing the present invention (surgeons, surgical assistants, etc.), the anastomotic device requires a short time to deploy and complete the anastomotic surgery. This is beneficial as it results in much shorter cardiopulmonary bypass times and ischemic times for the lower body in cases of ascending aortic aneurysms and descending aortic aneurysms (including abdominal aortic aneurysms), respectively.

Another advantage of the present invention is the minimally invasive approach available when deploying the anastomotic device, which can be used endoscopically, with or without a robot or other surgeon-directed machine. Further, the use of the present invention results in a time savings during an anastomotic surgery. This is beneficial as it eliminates bleeding, kidney failure, respiratory failure and ischemia (complications due to long bypass times). The risks of long surgery times during a thoracic aortic surgery include paralysis due to ischemia of the spinal chord.

### Second Embodiment

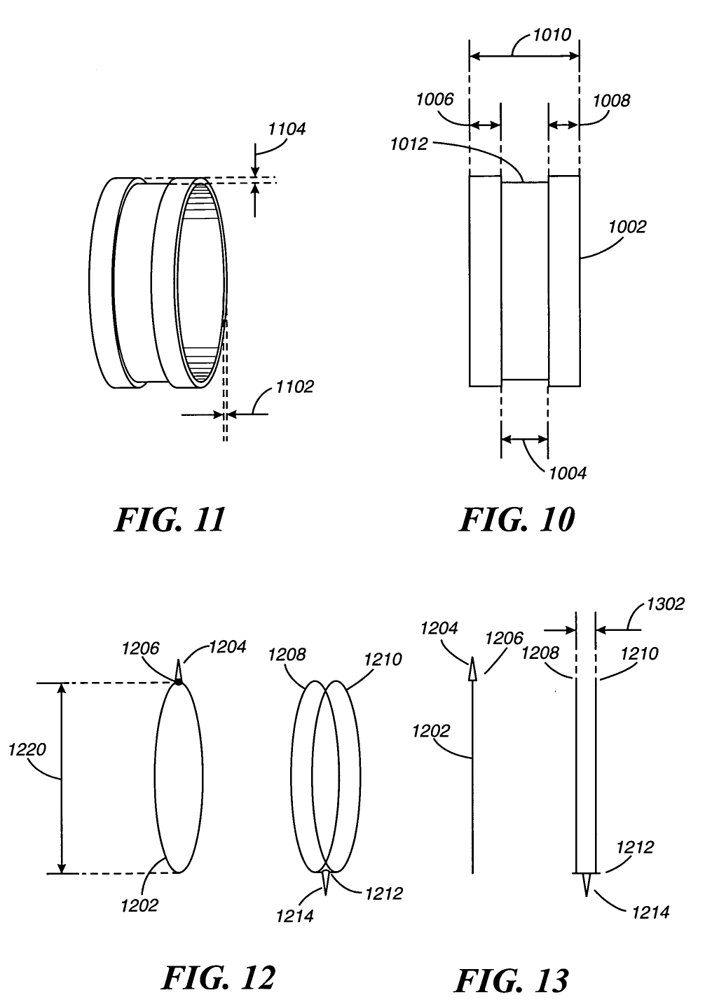

FIG. 10 is an illustration of a side view of a ring 1002 in a second embodiment of the present invention. The ring 1002 is another embodiment of the ring 100 depicted in FIG. 1. The ring 1002, having a tubular or cylindrical shape, includes a gutter 1012 along the outside surface of the ring 1002. Located to each side of the gutter 1012 is a portion of the ring 1002 with a larger outside diameter. Thus, the gutter 1012 has an outside diameter that is shorter in length than the outside diameters of the portions of the ring 1002 located to the sides of the gutter 1012. FIG. 10 shows that the gutter 102 has width 202 which can be from about 2 mm to about 5 mm. Further, the portions of the ring 1002 located to the sides of the gutter 1012 each have a width of 1006, 1008, respectively. Widths 1006, 1008 can be from about 1 mm to about 4 mm. The overall width 1010 of the ring 1002 (which is a sum of widths 1006, 1004 and 1008) can be from about 4 mm to about 13 mm.

The ring 1002, like ring 100, must be placed inside of the lumen of a tubular vascular element such as a vein, an artery, any other type of blood vessel or a prosthetic graft. Thus, the outside diameter of the ring 1002 (specifically the outside diameters of the portions of the ring 1002 located to the sides of the gutter 1012) must accommodate the inside diameter of the lumen of the vascular element in which the ring 1002 is placed, such that the ring 1002 fits securely within the lumen. That is, the outside diameter of the ring 1002 is substantially identical to, or slightly smaller than, the inside diameter of the lumen of the vascular element in which the ring 1002 is placed. Thus, the outside diameter of the ring 1002 can be from about 6 mm to about 45 mm, depending on th vascular element in which the ring 1002 is placed.

FIG. 11 is an illustration of an angled view of the ring 1002 of FIG. 10. FIG. 11 also shows the surface of the ring 1002 having a thickness 1102. The thickness 1102 of the surface of the ring 1002 can be from about 1 mm to about 4 mm. FIG. 11 also shows an angled view of the gutter 1012. Height 1104 represents the difference in the radius of the outside circumference of the gutter 1012 and the outside circumferences of the portions of the ring 1002 located to the sides of the gutter 1012. The height 1104 can be from about 0.5 mm to about 3 mm.

FIG. 11 further shows a lumen of the ring 1002, which must be substantially wide to allow a proper amount of blood to flow through the ring 1002. For this reason, the inside diameter of th ring 1002, i.e., the size of the lumen, can be from about 6 mm to about 40 mm.

FIG. 12 is an illustration of an angled view of a set of loops for placing around the ring of FIG. 10 in one embodiment of the present invention. FIG. 12 shows a single loop 1202 comprising a ring composed of metal or an alloy. Coupled to the loop 1202 is a spike 1204, similar in size, function and dimensions as the spike 108 of FIG. 1. The spike 1204 is coupled to the loop 1202 via a coupler 1206 which can be a metal or alloy element that is integrated with the spike 1204 and couples to the loop 1202. The diameter 1220 of the loop 1202 must accommodate the outside diameter of the gutter 1012 as the loop 1202 is placed around the ring 1002 within the gutter 1202 (see FIG. 16 below). Thus the diameter 1220 of the loop 1202 can be from about 6 mm to about 45 mm.

FIG. 12 further shows a double loop (loop 1208 and loop 1210) comprising two rings composed of the same metal or alloy as loop 1202. Coupled to the loops 1208 and 1210 are a spike 1214, similar in size, function and dimensions as the spike 110 of FIG. 1. The spike 1214 is coupled to the loops 1208, 1210 via a coupler 1212 which can be a metal or alloy element that is integrated with the spike 1214 and couples to the loops 1208, 1210. The diameter 1220 of the loops 1208, 1210 are identical to that of the loop 1202, and must accommodate the outside diameter of the gutter 1012 as the loops 1208, 1210 are placed around the ring 1002 within the gutter 1202 (see FIG. 16). Thus the diameter 1220 of the loops 1208, 1210 can be from about 6 mm to about 45 mm.

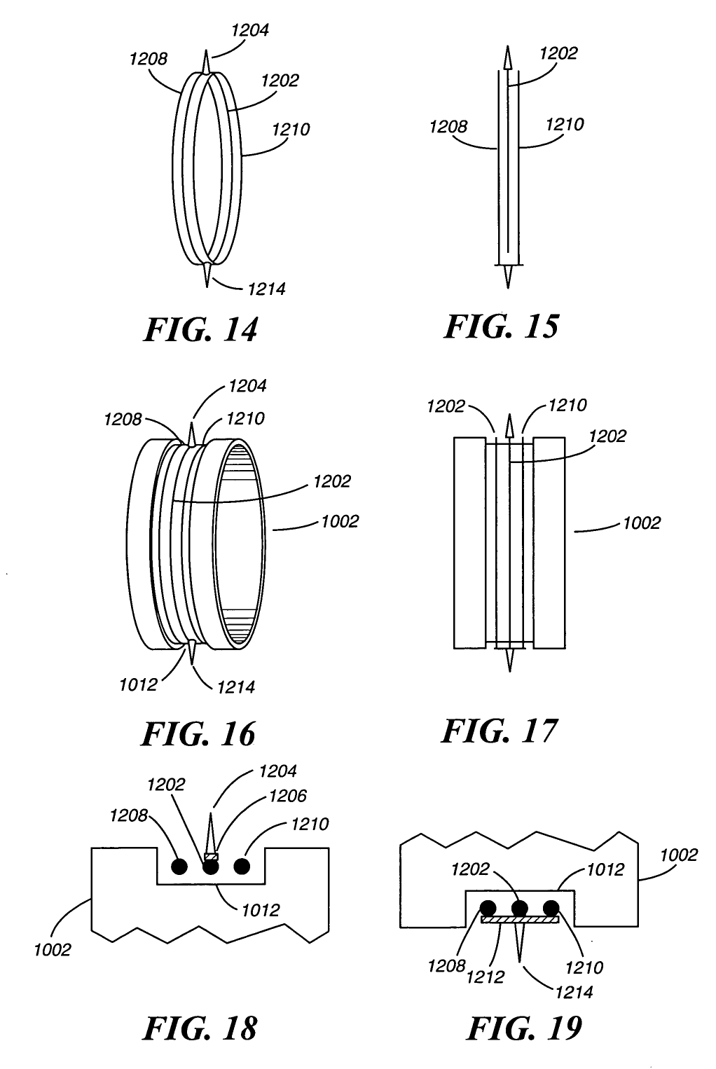

FIG. 13 is an illustration of a side view of the set of loops of FIG. 12. FIG. 13 shows the single loop 1202 with the spike 1204 coupled to th loop 1202 via a coupler 1206 FIG. 13 further shows the double loop (loop 1208 and loop 1210) with th spike 1214 coupled to the loops 1208, 1210 via a coupler 1212. FIG. 13 further shows a distance 1302 between loop 1208 and loop 1210. This distance 1302 allows the loop 1202 to be placed between loops 1208 and 1210 (see FIG. 14 below) such that loop 1202 and loops 1208 and 1210 are concentric. The distance 1302 can be from about 0.6 mm to about 3 mm.

FIG. 14 is an illustration of an angled view of the set of loops of FIG. 12 in an interlaced arrangement. FIG. 14 shows that the loop 1202 is placed between loops 1208 and 1210 such that loop 1202 and loops 1208 and 1210 are concentric. FIG. 14 further shows that that spikes 1204 and 1214 are located 180 degrees apart from each other around the circumference of the loops. In another embodiment of the present invention, spikes 1204 and 1214 are located any number of degrees apart from each other around the circumference of the loops. It should be noted that although the loops show only two spikes, the present invention supports any number of spikes located any number of degrees apart from each other around the circumference of the loops.

FIG. 15 is an illustration of a side view of the set of loops of FIG. 12 in an interlaced arrangement. FIG. 15 shows a side view of the loop 1202 being placed between loops 1208 and 1210 such that loop 1202 and loops 1208 and 1210 are concentric.

FIG. 16 is an illustration of an angled view of the ring 1002 of FIG. 10 coupled with the set of loops of FIG. 12. The loops 1202, 1208, 1210 are placed around the outside circumference of the gutter 1012 of the ring 1002 such that the loops 1202, 1208 and 1210 are concentric with the ring 1002. The diameter 1220 of the loops 1202, 1208, 1210 accommodate the outside diameter of the gutter 1012 of the ring 1002 as the loops 1202, 1208, 1210 are placed around the ring 1002 within the gutter 1202. Thus, the diameter 1220 of the loops 1202, 1208, 1210 is slightly larger than the outside diameter of the gutter 1012 of the ring 1002.

Note that the loops 1202, 1208 and 1210 can rotate freely around the ring 1002. This allows the spikes 1204, 1214 to be adjusted to any location around the ring 1002. The variable positioning of spikes in any location around the circumference of the ring 1002 is advantageous because it allows the spike(s) to be specifically positioned in places where they are needed. By allowing the variable positioning of the spikes around the ring 1002, diseased or damaged surface areas of the vascular element can be avoided and the spikes can be concentrated on healthy and/or structurally sound surface areas that would allow a spike to pierce the surface and secure the ring 1002 within the lumen of the vascular element.

FIG. 17 is an illustration of a side view of the ring 1002 of FIG. 10 coupled with the set of loops of FIG. 12. FIG. 17 shows a side view of the loops 1202, 1208, 1210 presently placed around the outside circumference of the gutter 1012 of the ring 1002 such that the loops 1202, 1208 and 1210 are concentric with the ring 1002.

FIG. 18 is an illustration of a cross sectional view of the top side of FIG. 17. FIG. 18 shows a cross section of the top side of the ring 1002, showing the gutter 1012. FIG. 18 also shows cross sections of the loops 1202, 1208 and 1210. Note that the loops 1202, 1208 and 1210 are located a short distance from the outside circumference of the gutter 1012 of the ring 1002. This allows the loops 1202, 1208 and 1210 to rotate freely around the ring 1002. Further, it is shown that the diameter 1220 of the loops 1202, 1208, 1210 is slightly larger than the outside diameter of the gutter 1012 of the ring 1002. FIG. 18 further shows the single loop 1202 with the spike 1204 coupled to the loop 1202 via a coupler 1206.

FIG. 19 is an illustration of a cross sectional view of the bottom side of FIG. 17. FIG. 19 shows a cross section of the bottom side of the ring 1002, showing the gutter 1012. FIG. 19 also shows cross sections of the loops 1202, 1208 and 1210. Further, it is shown that the diameter 1220 of the loops 1202, 1208, 1210 is slightly larger than the outside diameter of the gutter 1012 of the ring 1002. FIG. 19 further shows a cross section of the double loop (loop 1208 and loop 1210) with the spike 1214 coupled to the loops 1208, 1210 via a coupler 1212.

### Further Embodiments

In an embodiment of the present invention, the ring 100 includes orifices, such as orifices and 404, 406 and the circular strap 300 includes spikes, such as spikes 108, 110. In this embodiment, the circular strap 300 includes spikes that are located on the inside circumference of the circular strap 300 and extend radially inward toward the center of the lumen of the circular strap. The ring 100 includes orifices (aligned in position to the spikes of the circular strap) that extend through the surface of the ring 100.

In this embodiment, the ring 100 is placed within a first vascular element such as first vascular element 702, as shown in FIG. 7. Subsequently, the first vascular element 702 is placed within the lumen of the second vascular element 704, as shown in FIG. 8. Then, as shown in FIG. 9, the circular strap is placed around the second vascular element 704, wherein the spikes of circular strap pierce through the first vascular element 702 and the second vascular element 704 and extend through the orifices in the ring 100 so as to secure the circular strap 300 and the ring 100 within the lumen of the two vascular elements 702, 704.

In another embodiment of the present invention, the ring 100 or 1002 can also include an aortic valve, which is used for aortic valve replacement. The aortic valve is deployed in the usual manner in the aortic root under the ostia of the coronary arteries. In this embodiment, the aortic valve is located within the lumen of the ring 100 and is coupled to the inside circumference of the ring 100 so as to be held securely within the lumen of the ring 100.

In this embodiment, the anastomotic surgery occurs as follows. First, before arresting the heart, the aortopulmonary groove is dissected out proximal to the origin of the right coronary artery. The aortic wall is separated from the right ventricular outflow tract going in a more posterior direction towards the left sinus of the Valsalva. Taking care to stay proximal to the origins of both the right and the left coronary artery, the circular strap 300 is passed around the aortic root in the space created above.

Cardiopulmonary bypass is then initiated (If it has not already been initiated so far) and the heart is arrested by the conventional cardioplegia techniques. A standard aortotomy is then made and the aortic valve examined. If the valve must be replaced, then it is excised and the annulus debrided of calcium. The valve is then sized and an appropriate sized ring 100 or 1002 is then introduced into the aorta. The ring 100, including the aortic within it, is positioned below the origins of the coronary arteries and the spikes are traversed through the aortic wall at appropriate points around the circumference. It should be assured at this time that the valve leaflets comprising the aortic valve move freely and that they do not obstruct the origins of the coronary arteries. Thus deployed, the spikes are fixed at both ends to the ring 100 and the strap 300, which is then tightened around the inner ring. The valve is tested and the aortotomy closed in the usual fashion. The operation is finished using conventional techniques of deairing and weaning off of cardiopulmonary bypass.

### Conclusion

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

What is claimed is:

1. An apparatus for facilitating anastomosis of a first tubular element with a second tubular element, comprising:
a ring for placement inside of the first tubular element such that the ring and the first tubular element are concentric;
a circular strap for placement around the second tubular element such that the circular strap and the first tubular element are concentric, wherein a first end of the first tubular element is placed inside of a first end of the second tubular element such that the ring and the circular strap are concentric; and
an adjustment element coupled to the circular strap for adjusting the circular strap to apply pressure onto the ring and secure the first end of the first tubular element to the first end of the second tubular element.

2. The apparatus of claim 1, further comprising:
at least one spike coupled to the outside diameter of the ring, wherein when the ring is placed inside of the first tubular element, the at least one spike extends through a surface of the first tubular element so as to secure the ring inside of the first tubular element.

3. The apparatus of claim 2, wherein the at least one spike extends through a surface of the second tubular element when the first tubular element is placed inside of the second tubular element, so as to secure the first tubular element inside of the second tubular element.

4. The apparatus of claim 2, further comprising:
at least one orifice in the circular strap, wherein the at least on orifice is aligned with the at least one spike when the first tubular element is placed inside of the second tubular element such that the at least one spike extends through the at least one orifice, so as to secure the ring inside of the circular strap.

5. The apparatus of claim 1, wherein the first tubular element and the second tubular element are any one of:
a vein;
an artery; and
a vascular prosthesis.

6. The apparatus of claim 5, wherein the first tubular element is a graft.

7. The apparatus of claim 1, wherein the ring is substantially the same diameter as the inside diameter of the first tubular element and the circular strap is substantially the same diameter as the outside diameter of the second tubular element.

8. The apparatus of claim 1, wherein the ring is placed inside of the first tubular element substantially near the first end of the first tubular element and the circular strap is placed around the second tubular element substantially near the first end of the second tubular element.

9. The apparatus of claim 1, wherein the adjustment element coupled to th circular strap is any one of:
a ratcheting element;
a buckling element; and
a screwing element.

10. The apparatus of claim 1, wherein the ring is composed substantially of any one of a metal and an alloy.

11. An apparatus for facilitating anastomosis of a first tubular element with a second tubular element, comprising:
a ring for placement inside of the first tubular element such that the ring and the first tubular element are concentric;
at least one adjustable spike coupled to the outside diameter of the ring, wherein when the ring is placed inside of the first tubular element, the at least one spike extends through a surface of the first tubular element so as to secure the ring inside of the first tubular element and wherein the location of the at least one adjustable spike on the outside diameter of the ring can be adjusted;
a circular strap for placement around the second tubular element such that the circular strap and the first tubular element are concentric, wherein a first end of the first tubular element is placed inside of a first end of the second tubular element such that the ring and the circular strap are concentric; and
an adjustment element coupled to the circular strap for adjusting the circular strap to apply pressure onto the ring and secure the first end of the first tubular element to the first end of the second tubular element.

12. The apparatus of claim 11, wherein the at least one spike extends through a surface of the second tubular element when the first tubular element is placed inside of the second tubular element, so as to secure the first tubular element inside of the second tubular element.

13. The apparatus of claim 11, further comprising:
at least one orifice in the circular strap, wherein the at least one orifice is aligned with the at least one spike when the first tubular element is placed inside of the second tubular element such that the at least one spike extends through the at least one orifice, so as to secure the ring inside of the circular strap.

14. The apparatus of claim 11, wherein the first tubular element and the second tubular element are any one of:
a vein;
an artery; and
a vascular prosthesis.

15. The apparatus of claim 14, wherein the first tubular element is a graft.

16. The apparatus of claim 11, wherein the ring is substantially the same diameter as the inside diameter of the first tubular element and the circular strap is substantially the same diameter as the outside diameter of the second tubular element.

17. The apparatus of claim 11, wherein the ring is placed inside of the first tubular element substantially near the first end of the first tubular element and the circular strap is placed around the second tubular element substantially near the first end of the second tubular element.

18. The apparatus of claim 11, wherein the adjustment element coupled to the circular strap is any one of:
a ratcheting element;
a buckling element; and
a screwing element.

19. The apparatus of claim 11, wherein the ring is composed substantially of any one of a metal and an alloy.

20. The apparatus of claim 11, wherein the at least one spike is composed substantially of any one of a metal and an alloy.
